(19) United States
(12) Patent Application Publication
(10) Pub. No.: US 2009/0036908 A1
(43) Pub. Date: Feb. 5, 2009

(54) Biologic Barrier for Implants That Pass Through Mucosal or Cutaneous Tissue

(76) Inventors: Ron Zokol, Vancouver (CA); Ian Dickey, Bangor, ME (US)

Correspondence Address: HANCOCK HUGHEY LLP, P.O. BOX 1208, SISTERS, OR, 97759, US

(21) Appl. No.: 11/886,805

(22) PCT Filed: Mar. 30, 2005
(86) PCT No.: PCT/US2005/011219

§ 371 Date: Sep. 19, 2007

(51) Int. Cl. A61B 17/08; A61F 2/28

(52) U.S. Cl. 606/151; 623/16.11

(57) ABSTRACT

Apparatus is described for creating a direct mechanical connection between skeletal bone and a prosthetic device located outside of the body. The apparatus provides a means for creating an effective biologic seal to prevent the transmission of microbiologic particles into the body.

### FIELD OF THE INVENTION

The present invention relates generally to the medical field, and more particularly to apparatus for creating a direct mechanical connection between skeletal bone and a structure located outside overlying soft tissues, wherein the mechanical connection passes through the overlying soft tissues and creates a biologic barrier between the inside and the outside of the overlying tissues, or between the bone and the outside of the body.

### BACKGROUND OF THE INVENTION

In the treatment of certain medical conditions it is desirable to provide a mechanical connection between an external structure and the patient's skeletal bone. Examples include external prostheses, such as artificial limbs or teeth, and external fixation pins used in reconstructive orthopedics. In each of these cases, it is desirable to create a direct mechanical linkage between a device external to the body and bone inside the body. This linkage can be characterized as having three functional components: a first part that is anchored to the bone; a second part that passes through the mucosal or cutaneous surface of the body; and a third part that is mechanically linked to the prosthesis or other device outside of the body.

Various devices intended for fixation to bone are known in the art, including artificial joint replacements, metal plates used to repair bone, anchors for attaching ligaments or tendons to bone, and dental implants. Typical examples of such devices are the OmniFit™ EON® hip joint replacement (Stryker Orthopedics, Mahwah N.J.), the NexGen® knee joint replacement, the Statak® soft tissue attachment anchor, (Zimmer Inc, Warsaw Ind.), and various dental implants (Nobel Biocare AB, Gothenburg Sweden; Institut Straumann AG, Waldenberg Switzerland; Zimmer Dental Inc, Carlsbad Calif.; Dentsply International Inc., York, Pa.; Biohorizons, Birmingham Ala.).

Three different techniques are commonly used to fix devices to bone: cement; mechanical interconnections using friction (screws, staples); and materials that promote growth of bone into the device. The Duracon® knee joint (Stryker) is an example of a device that is fixed to bone with cement, wherein the cement interlocks with the surface structure of the bone and with the surface structure of the implant to provide fixation. The NaturalHip T™ (Zimmer Inc) is an example of a device that has a surface intended to promote bone growth into the implant, creating a direct connection between the bone and the implant. Most dental implants and soft tissue anchors use some variation of screw intended to thread directly into the bone.

All of these techniques, when properly used, provide a reliable mechanical connection between the prosthetic device and bone.

There are many techniques suitable for connecting a prosthetic to an implant, including interference fits, tapers, threads, cements or adhesives. Dental implants typically use threads or cement to attach the ceramic or metal and ceramic to the part of the implant fixed to the bone.

Implants that pass through the mucosal or cutaneous tissues (such as dental implants, percutaneous access devices or orthopedic external fixation pins) offer a significant risk of infection. Tissue in contact with the implant can usually be disrupted with minimal force, providing a channel through which infectious microorganisms may pass into the body.

It is well known that the stimulation of fibrous in-growth into an implant where the implant passes through soft tissue to the outside of the body can reduce infection. The presence of attached tissue, (such as gingival tissue in the case of a dental implant) significantly improves the biologic seal around the implant, thereby inhibiting the access of microorganisms to the tissue and bone around the implant. It is also recognized that increased blood supply to the tissue near the implant leads to increased white blood cell concentration and a commensurate reduction of infection around the implant site. Furthermore, in the case of dental implants, promotion of fibrous in-growth to the implant more closely mimics the natural connection between the tooth and jaw.

A suitable means for promoting the in-growth into that portion of an implant that passes through mucosal or cutaneous tissue is to construct the implant, or at least that part of the implant intended to be in contact with tissue, of a material that promotes such in-growth. It is known in the art to provide such a means in the case of percutaneous access devices (e.g. U.S. Pat. No. 4,897,081), through the use of biocompatible polymer material. Although this approach is suitable for relatively short-term applications, such polymers do not offer the high strength and long life required in the case of implants that provide a mechanical linkage between the skeleton and a device outside the body.

It is also known in the art to provide an implant with a region of porous metal into which soft tissue can grow. For example, U.S. Pat. No. 3,855,638 discloses a porous region comprising small, discrete particles of metallic material bound together at their points of contact for promoting such tissue ingrowth. Examples of porous metallic materials described in this patent include austenitic stainless steel, titanium, titanium alloys and cobalt alloys, and the cobalt alloy called Vitallium™.

There remains a need for improved materials and apparatus for promoting tissue ingrowth in connection with implant devices.

### SUMMARY OF THE INVENTION

The subject invention provides an implant intended for connecting a device located outside the body to skeletal bone, including a means for promoting the in-growth of tissue into that portion of the implant that passes through mucosal or cutaneous tissues, in order to provide a biologic seal around the implant. The means for promoting in-growth consists of providing a region of trabecular metal, such as titanium or tantalum, in that portion of the surface of the implant that is intended to come into contact with mucosal or cutaneous tissues.

In another aspect of the invention, means is provided to remove and replace that portion of the implant disposed to promote the in-growth of tissue, such that the in-grown portion can be removed and replaced should it become infected, without the need to remove the entire implant.

An implant in accordance with the invention may take several forms. A dental implant in accordance with the invention provides a separate, removable section of trabecular metal connected to a first portion intended for connection to bone and removeably connected to a second portion intended for connection to a tooth prosthesis. An orthopedic external fixation pin in accordance with the invention provides a region of trabecular metal where the pin passes through the cutaneous tissue. An implant for connecting a limb prosthesis to a person's body in accordance with the invention provides a portion disposed for connection to bone, a second portion disposed for connection to a limb prosthesis, and a region of trabecular metal where the implant passes through the cutaneous tissue.

In an alternative embodiment, a dental implant in accordance with the invention provides a section of trabecular metal intended for connection to gingival and connective tissue, connected to a portion intended for connection to a tooth prosthesis. In this alternative embodiment, the dental implant does not connect directly with the bone, but instead is held in place by attachment of the underlying tissues, advantageously mimicking the fixation of natural teeth.

Trabecular metals are utilized in accordance with the current invention to promote tissue ingrowth. Trabecular metals provide an approximately dodecahedral pore structure, having highly connected (“open cell”) porosity that promotes a high degree of tissue linkage through the pores, intertwining the ingrown tissue with the trabecular metal structure. This provides a strong connection between the soft tissue and the metallic structure and promotes vascularization of the ingrown tissues.

An implant that disposes a material a trabecular metal in the region of the implant that passes through gingival, mucosal or cutaneous tissue promotes strongly attached fibrous in-growth of the tissue, thereby reducing the risk of infection of the underlying tissue and bone.

### BRIEF DESCRIPTION OF THE DRAWINGS

The foregoing and other objects, features and advantages of the present invention will become apparent upon reference to the following detailed description of the preferred embodiments and to the drawings, wherein:

FIG. 1 is a representation of a dental implant in accordance with the invention.

FIG. 2 is a cross-section illustration of the dental implant of FIG. 1 as it would appear when implanted.

FIG. 2A is a cross-section illustration of an alternate embodiment of the dental implant of FIG. 1 and FIG. 2.

FIG. 3 is a cross-section illustration of an another alternate embodiment of the dental implant of FIG. 1 and FIG. 2.

FIG. 3A is an alternate embodiment of the dental implant of FIG. 3.

FIG. 4 is an illustration of an orthopaedic external fixation pin in accordance with the invention.

FIG. 5 is an illustration of an implant intended for connecting an artificial limb to a person's skeleton.

### DETAILED DESCRIPTION OF THE PREFERRED EMBODIMENTS

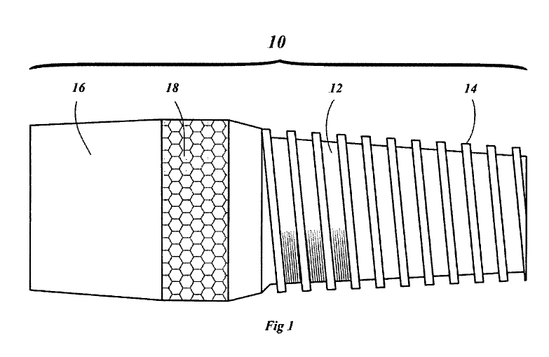

With reference to FIG. 1, an embodiment of the invention is illustrated in the form of a dental implant 10. Dental implant 10 is a generally cylindrical object made of a metal such as stainless steel, titanium, tantalum or an alloy of titanium and tantalum and similar metals. Proximal portion 12 of implant 10 provides features to promote secure attachment to bone. In FIG. 1, bone attachment is facilitated with screw thread 14; however, other means for attaching implant 10 to bone are possible without departing from the current invention. Other means for attaching implant 10 to bone include the provision of a convoluted region for attachment of cement, or provision of a region of trabecular metal for promoting the in-growth of bone.

Distal portion 16 of implant 10 provides for attachment of a prosthetic tooth. Such attachment is achieved with the use of cement, although other attachment means may be used, such as an interference fit, taper fit, threading or other means.

Intermediate portion 18 of implant 10 is made of trabecular metal, preferably titanium or tantalum, having pore sizes in the range of 100 to 800 microns. In the preferred embodiment, the trabecular metal is formed directly on implant 10 using a chemical vapour deposition technique that allows deposition of trabecular metal on selected regions of a substrate. (e.g. Hedrocel™ Implex Corporation, Allendale, N.J.), Other techniques for forming trabecular metal are known in the art, such as carbon dioxide injection (U.S. Pat. No. 6,759,004), moulding (e.g. U.S. Pat. No. 6,221,447 or U.S. Pat. No. 5,958,314) or sintering (U.S. Pat. No. 6,674,042). As noted earlier, trabecular metals such as those advantageously used herein provide an approximately and substantially dodecahedral pore structure, having highly connected open cell porosity that promotes a high degree of tissue linkage through the pores, intertwining the ingrown tissue with the trabecular metal structure. This provides a strong connection between the soft tissue and the metallic structure and promotes vascularization of the ingrown tissues.

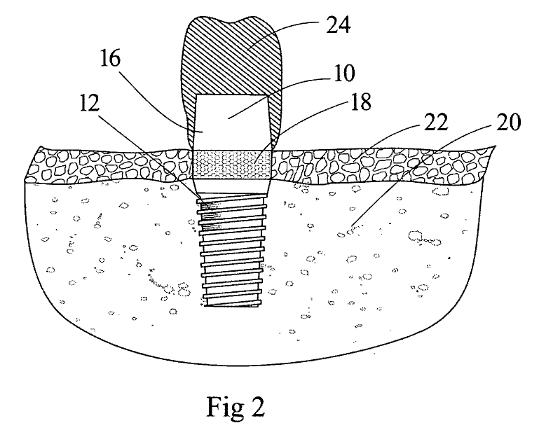

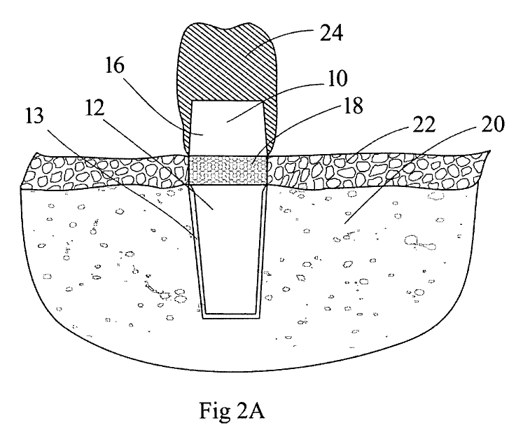

Referring to FIG. 2, proximal portion 12 of implant 10 is embedded into bone 20, such that intermediate portion 18 is in contact with gingival tissue 22. Prosthetic tooth 24 (or other dental bridge work) is attached to distal portion 16 of implant 10. Gingival tissue 22 grows into the trabecular metal that makes up intermediate portion 18, creating a circumferential, neo-vascularized biologic seal around implant 10. As used herein, the terms “biologic seal” and “biologic barrier” refer to a condition that mimics or approximates the barrier conditions to microbiological invasion that exist in normal, undisturbed tissue of a similar type. Thus for example, the term biologic seal is not meant to characterize an absolute barrier to microbiologic organisms crossing between external tissues and internal tissues. Instead, the term refers to a condition at the area of tissue ingrowth that inhibits microbiologic invasion, but which does not necessarily define an absolute barrier.

As illustrated in FIG. 2, the intermediate portion 18 is in contact with tissue between bone 20 and the external surface of the tissues and the tissue ingrowth into the intermediate portion provides an effective biologic barrier to inhibit invasion of microbiologics into internal tissues. Stated another way, intermediate portion 18 extends through the interface between internal tissues and the external surface of overlying tissues.

FIG. 2A is an alternate embodiment of the dental implant of FIG. 1 and FIG. 2, which has the advantage of mimicking the fixation of natural teeth. In this embodiment, proximal portion 12 of implant 10 is loosely fitted into socket 13 formed in bone 20. Implant 10 is held in place by the in-growth of gingival tissue into intermediate portion 18. This provides a fixation that permits prosthetic tooth 24 to move slightly with respect to bone 20 in response to externally applied forces. Such motion serves to transfer mechanical loads on tooth 24 to adjacent natural or prosthetic teeth, reducing the likelihood of mechanical failure.

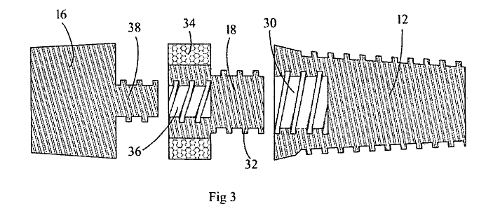

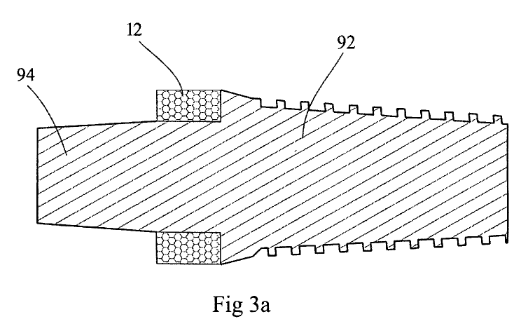

FIG. 3 illustrates a longitudinal cross section view of an alternate embodiment of dental implant 10. In this embodiment, distal portion 12 of implant 10 is a separate piece, having a threaded socket 30. Intermediate portion 18 has a threaded stud 32 which mates with socket 30. It will be appreciated that the socket 30 and stud 32 may be fastened together in other ways, including for example by cementation, mechanical thread interlock or other means. The periphery of intermediate portion 18 is made of trabecular metal 34. Intermediate portion 18 also includes socket 36, to which stud 38 of distal component 16 may be connected and fastened in place using cement, threads or other means. This alternate embodiment provides the option for removal of distal component 16 from intermediate component 18. This permits the detachment of component 18 from any in-grown gingival tissue with minimal gingival tissue loss, the removal of intermediate component 18 from proximal component 12, and the replacement of intermediate component 18 with a new component or temporary spacer for subsequent tissue management. This may be required if intermediate component 18 becomes seeded with bacteria and is infected.

FIG. 3A shows a longitudinal cross section of an alternate embodiment of the dental implant of FIG. 3. In this embodiment, the implant is made of a first part having proximal end 92 disposed for fixation to bone and a distal part 94 disposed for connection to a prosthetic tooth or bridgework. Annular ring 90, made of trabecular metal is removeably fitted around distal part 94. This permits detachment of ring 90 from any in-grown gingival tissue with minimal gingival tissue loss, the removal of ring 90, and the replacement of ring 90 with a new component or temporary spacer for subsequent tissue management. Advantageously, this alternate embodiment of the dental implant provides a single mechanical link between the prosthesis and the bone into which the dental implant is inserted.

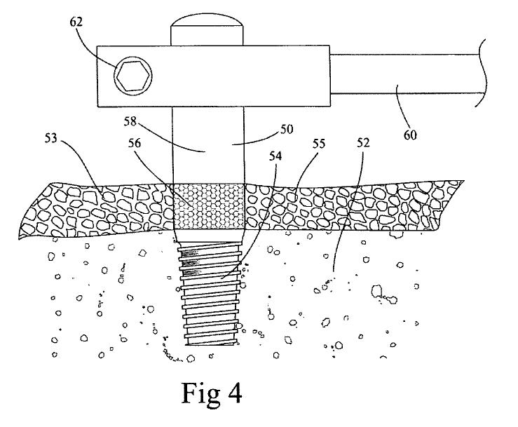

FIG. 4 illustrates an orthopaedic external fixation pin according to the invention, shown as it might be implanted for use. Fixation pin 50 is anchored into bone 52 at proximal end 54, such that intermediate portion 56 is in contact with cutaneous and subcutaneous tissue 55 and underlying muscle tissue 53. Intermediate portion 56 is made of trabecular metal, preferably titanium or tantalum. Distal portion 58 of fixation pin 50 is disposed for connection to external fixation device 60 with connection means 62.

Once implanted as shown, cutaneous and subcutaneous tissue 53 grows into the trabecular metal of intermediate portion 56, creating a biological seal.

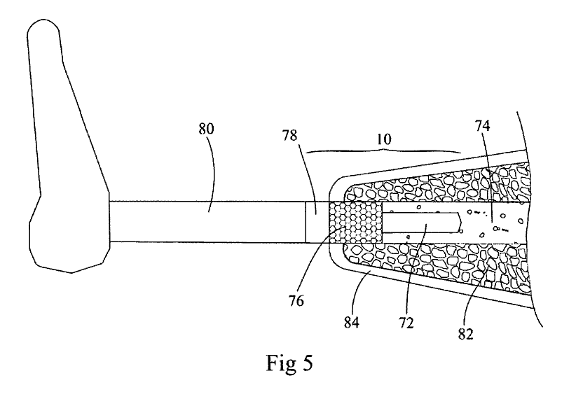

FIG. 5 shows a partial para-sagital section of an implant for connecting a prosthesis to an amputated limb. Implant 70 is made up of proximal portion 72, which is rigidly connected to bone 74 with cement or other fastener system as described above Intermediate portion 76 of implant 70 is made of trabecular metal preferably titanium or tantalum. Distal portion 78 of implant 70 is disposed for rigid connection to artificial limb 80.

When implanted as shown, cutaneous and sub-cutaneous tissue 84 and muscle tissue 82 grow into intermediate portion 76, forming a biologic seal.

Many different configurations of the implant apparatus described herein may be constructed without departing from the scope and spirit of the present invention, therefore the present invention should be limited only by the scope of the appended claims. For example, an implant in accordance with the invention could be an entirely trabecular metal component having an attachment means for the external prosthesis. In another possible embodiment, a dental implant in accordance with the invention could be constructed largely of materials other than metal, while providing that the portion of the implant passing through the gingival tissue is made of a material that promotes in-growth of gingival tissue.

We claim:

1. Apparatus for making a direct mechanical connection between skeletal bone and a prosthesis located externally of tissues overlying the bone, comprising:
a proximal portion configured for fixation to bone;
a distal portion configured for fixation to a prosthesis; and
an intermediate portion between the proximal and distal portions and configured to contact tissue overlying the bone, said intermediate portion made at least in part of a trabecular metal formed of interconnected pores having a substantially dodecahedral geometry, and wherein said intermediate portion is configured for promoting ingrowth of tissue into said intermediate portion to promote an effective biologic seal where said intermediate portion is in contact with said tissue.

2. The apparatus according to claim 1 in which the trabecular metal is titanium.

3. The apparatus according to claim 1 in which the trabecular metal is tantalum.

4. The apparatus according to claim 1 in which the trabecular metal is an alloy of titanium and tantalum.

5. The apparatus according to claim 1 in which the intermediate portion is removable from the proximal portion.

6. Apparatus for attaching an external prosthetic device to skeletal bone, comprising:
an external portion configured for attachment to the prosthetic device externally of tissue;
an internal portion configured for attachment to skeletal bone; and
an intermediate portion configured for interconnecting the internal and external portions and passing through tissue between said skeletal bone and overlying tissue, said intermediate portion made at least in part of a metal that promotes ingrowth of tissue into said intermediate portion to thereby form an effective biologic barrier where the intermediate portion extends externally through said tissue.

7. The apparatus according to claim 6 in which the intermediate portion comprises a trabecular metal.

8. The apparatus according to claim 7 in which the intermediate portion comprises interconnected pores defining a substantially dodecahedral geometry.

9. The apparatus according to claim 7 in which the trabecular metal comprises titanium.

10. The apparatus according to claim 7 in which the trabecular metal comprises tantalum.

11. The apparatus according to claim 6 in which the external prosthetic device comprises a dental implant.

12. The apparatus according to claim 11 in which the dental implant is removable from the external portion and wherein the intermediate portion is removable from the internal portion.

13. A method of attaching an external prosthesis to skeletal bone, comprising the steps:
(a) attaching a first member to skeletal bone;
(b) attaching a second member to the first member in a position such that the second member extends through tissue overlying said skeletal bone and such that a distal end of said second member is positioned externally of said tissue so that an external prosthetic may be attached to said distal end, said second member comprising at least in part trabecular metal defining interconnected pores;
(c) attaching an external prosthesis to the distal end of said second member.

14. The method according to claim 13 including the step of promoting ingrowth of tissue into said second member to form an effective biologic seal around said second member.

15. The method according to claim 14 including the step of providing the second member comprising titanium.

16. The method according to claim 13 wherein the external prosthesis is a dental implant.

17. An orthopaedic fixation pin comprising a proximal member configured for fixation to bone, a distal member configured for fixation to an external frame for fixation to an orthopaedic device, and an intermediate member configured for extending through the interface between internal and external tissues, wherein said intermediate member promotes ingrowth of tissue into said intermediate member.

18. The orthopaedic fixation pin according to claim 17 in which the intermediate member is detachable from said proximal member.

19. The orthopaedic fixation pin according to claim 18 in which the intermediate member is made at least in part of a trabecular metal.

20. The orthopaedic fixation pin according to claim 19 in which the trabecular metal comprises titanium.
